(19) United States
(12) Patent Application Publication
(10) Pub. No.: US 2006/0134212 A1
(43) Pub. Date: Jun. 22, 2006

(54) Lercanidipine immediate release compositions

(75) Inventors: Mahendra G. Dedhiya, Pomona, NY (US); Suneel K. Rastogi, Ballwin, MO (US); Anil Chhettry, Holtsville, NY (US)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/218,820

(22) Filed: Sep. 2, 2005

Related U.S. Application Data
(60) Provisional application No. 60/606,592, filed on Sep. 2, 2004.

(51) Int. Cl. A61K 31/445; A61K 9/24

(52) U.S. Cl. 424/472; 514/317

(57) ABSTRACT

The present invention provides an immediate release composition for the low solubility drug, lercanidipine. The immediate release composition of the present invention comprises a core; a first layer, comprising lercanidipine, a surfactant and a binder, and optionally, a second layer comprising a film coating.

### CROSS-REFERENCE TO RELATED APPLICATIONS

The present application claims the benefit of priority under 35 U.S.C. § 119(e) of Provisional Application Ser. No. 60/606,592, filed Sep. 2, 2004, which is hereby incorporated herein by reference in its entirety.

### FIELD OF THE INVENTION

The present invention relates to an immediate release pharmaceutical composition that achieves rapid release of lercanidipine, has sufficient bioavailability to impart a therapeutic effect and can be combined with additional active agents including modified release pharmaceutical compositions of lercanidipine to achieve a dosage form with predetermined multi-phase release and pharmacokinetic profile.

### BACKGROUND OF THE INVENTION

Solid oral drug compositions or preparations have various release profiles such as an immediate release profile as referenced by FDA guidelines (“Dissolution Testing of Immediate Release Solid Oral Dosage Forms”, issued 8/1997, Section IV-A) or an extended release profile as referenced by FDA Guidelines (“Extended Release Oral Dosage Forms: Development, Evaluation, and Application of In Vitro/In Vivo Correlations”, Food and Drug Administration, CDER, September 1997, Page 17). For example, in the dissolution testing guideline for immediate release profiles, materials which dissolve at least 80% in the first 60 minutes, or in the first 30 minutes, an aqueous medium qualify as immediate release profiles.

The immediate release solid dosage forms are wherein the active ingredient is released over a short duration, such as 60 minutes or less, and the absorption of the drug is rapid. The time to reach maximum concentration (Tₘₐₓ) of drug in the body fluid is one of the parameters used to study drug absorption. Modified release solid oral dosage forms are products wherein for example, the active ingredient is released over an extended period of time in an effort to maintain therapeutically effective plasma levels over a similarly extended time interval and/or to affect other pharmacokinetic properties.

Various components having different release characteristics may be employed to yield compositions having a multiphase release profile, such as a portion of drug releasing immediately, followed by an extended release, to attain more specific therapeutic objectives.

Immediate release oral dosage forms have been described previously for hundreds of active agents, and include a number of compositions designed to provide rapid and immediate release of an active agent. Such dosage forms typically involve the use of one or more of the following methods: reducing the size of the active agent particles by micronization (see, e.g., U.S. Pat. No. 6,410,054); combining a micronized active agent with emulsifying agents (see, e.g., U.S. Pat. No. 4,892,741); and combining an active agent with hydrophilic and/or lipophilic surfactants (see, e.g., U.S. Pat. No. 6,569,463). The immediate release oral dosage forms of the prior art, however, have certain limitations, particularly when the active agent is poorly soluble. Therefore, there remains a need in the art for immediate release dosage forms containing low solubility drugs and in particular immediate release dosage forms containing the poorly soluble drug, lercanidipine.

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with a long duration of action and high vascular selectivity. The molecular formula of the hydrochloride salt of lercanidipine is set forth in Formula (I) below.

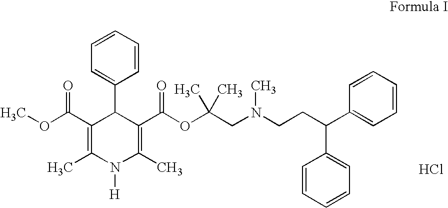

The hydrochloride salt of lercanidipine is commercially available from Recordati S.p.A. (Milan, Italy). Methods of making both lercanidipine free base and its hydrochloride salt have been described previously along with methods of resolving lercanidipine into individual enantiomers in U.S. Pat. Nos. 4,705,797; 5,767,136; 4,968,832; 5,912,351; and 5,696,139, all of which are incorporated herein by reference. Lercanidipine is a dihydropyridine calcium antagonist. As other calcium channel antagonists, it lowers blood pressure by relaxing arteriolar smooth muscle, which decreases peripheral vascular resistance. Lercanidipine produces no negative cardiac inotropism and, occasionally only, mild reflex tachycardia generally of short duration. It has a high affinity for and competitively antagonizes the dihyropyridine subunit of the L-type calcium channel. Lercanidipine has been approved for the treatment of hypertension and has been marketed in several European countries under the trademark Zanidip® since 1996.

Lercanidipine alone or in combination with additional active agents has been shown to be effective in once and twice daily administration. Lercanidipine has been studied in the dosage ranging from 2 to 80 mg. Lercanidipine is normally administered in a dosage of about 10 mg to about 20 mg once or twice daily, the recommended maximum dose being about 30 mg once or twice daily, all available in immediate release tablet form. Lercanidipine is used for treating mild to moderate hypertension and is also expected to be useful in alleviating angina pectoris. It has also been beneficial in elderly patients with isolated systolic hypertension. The recommended starting oral dose of lercanidipine is given by mouth 10 mg once daily before food and is increased, if necessary, after at least 2 weeks to 20 mg daily. Upon oral administration, lercanidipine is absorbed and peak plasma level occurs 1-3 hours following dosage.

Lercanidipine and its salts, such as the hydrochloride salt, are practically insoluble in water, displaying an aqueous solubility of about 5 μg/ml. The Lercanidipine is also practically insoluble in acidic media although it has marginally greater insolubility in acidic mediums. Even at pH 5 and above, its solubility is less than 20 μg/ml. Lercanidipine also shows low permeability (i.e., poor permeability with Pₐₚₚ of about 0.5×10⁻⁷ cm/s in a Caco-2 cell apparatus and low bioavilability) and is classified as a low permeable drug, as defined by the FDA. Additionally, when administered to patients, lercanidipine displays extensive presystemic first pass elimination as a result of its being a substrate for cytochorme P450 IIIA4 isoenzyme. The combination of poor water solubility, low permeability and considerable first pass metabolism results in low and highly variable bioavailability.

In order to improve the bioavailability of lercanidipine, food is co-administered with each dosage. The administration of food along with lercanidipine has been shown to increase the absorption of lercanidipine significantly and therefore enhance its efficacy, a phenomenon known as “food effect.” Studies have shown that simultaneous intake of food (especially food having a high fat content) increases the amount of lercanidipine absorbed between three and four times compared to administration without food. The same studies have shown that lercanidipine administered in the absence of food is not entirely absorbed which results in low and variable bioavailability. The dependence of effective dosing and absorption of lercanidipine upon co-administration of food is inherently undesirable and can result in fluctuations in effectiveness, inter-patient variability, and in poor patient acceptance and/or compliance.

Accordingly, in order to overcome one or more of the foregoing problems, increase the effectiveness of lercanidipine in patients, and provide for more predictable performance of this drug, there is a need in the art for an oral dosage form which affords improved absorption and bioavailability of lercanidipine at a lower maximum plasma concentration Cₘₐₓ. Particularly, there is a need for an oral dosage form that permits lercanidipine to be administered and absorbed, while reducing or eliminating the food effect. More particularly, there is a need for an immediate release pharmaceutical composition that itself provides rapid absorption of lercanidipine and can also be combined with modified release bead compositions containing lercanidipine and optionally with additionally active agents to form oral dosage forms with predetermined multiphase release and pharmacokinetic profiles.

### SUMMARY OF THE INVENTION

It has been found that an immediate release pharmaceutical composition can be prepared which provides rapid dissolution of the low solubility drug, lercanidipine. The pharmaceutical composition of the present invention provides rapid release of an effective amount of lercanidipine and at the same time increased permeability resulting in improved absorption while retaining an immediate release Tₘₐₓ characteristic over a dose range of 2 to 80 mg. Administration of the pharmaceutical composition of the present invention results in rapid onset of relief from hypertension. The composition of the present invention can be used alone or to prepare modified release dosage forms.

One embodiment of the present invention provides an immediate release pharmaceutical composition comprising: a core; a first layer comprising lercanidipine, a surfactant and a binder; and optionally a second layer comprising a film coating. Optionally, the pharmaceutical composition may be formed as immediate release beads, wherein the beads have a average radius from about 0.1 mm (140 mesh) to about 2 mm. (10 mesh).

Another embodiment of the present invention provides a novel lercanidipine immediate release bead composition in which the lercanidipine is present in the amount sufficient to provide a therapeutic effect when the composition is administered to a patient, e.g., from about 2 to about 80 mg lercanidipine.

In an additional embodiment the present invention provides immediate release solid dosage forms comprising lercanidipine that provide for the in vitro dissolution of more than about 80% of the lercanidipine within the first 60 minutes. The preferred method of evaluating the dissolution rate is the USP basket method at 100 RPM in 900 ml aqueous buffer 0.01N HCl, at 37° C.

In other embodiments the immediate release oral dosage forms of the present invention preferably provide a time to maximum plasma concentration (Tₘₐₓ) of about 1 to about 3 hours and a maximum plasma concentration (Cₘₐₓ) of lercanidipine of about 12 ng/mL for a 40 mg dose of lercanidipine.

Preferably the immediate release composition of the present invention comprise beads that may be combined and packaged in a capsule to create a solid oral dosage form. In another embodiment of the present invention, the immediate release composition comprise beads that may be combined with an additional excipient and compressed into a tablet to create a solid oral dosage form. If administered as an immediate release dosage form, the dosage form will contain a sufficient amount of immediate release beads to provide from about 2 to about 80 mg of lercanidipine per dose.

These and other aspects of the present invention will be apparent to those of ordinary skill in the art in the light of the present description, claims and figures.

### DESCRIPTION OF THE DRAWINGS

FIG. 1 depicts the dissolution profile of one composition comprising immediate release lercanidipine pharmaceutical compositions of the present invention.

FIG. 2 depicts the effect of surfactant on the dissolution profile of immediate release lercanidipine pharmaceutical bead compositions of the present invention, the curve represented by -▪- depicts the dissolution profile of an immediate release composition comprising Polysorbate 80 (Tween 80) as a surfactant, the curve represented by -♦- depicts the dissolution profile of an immediate release composition without surfactant.

FIG. 3 depicts the effect of surfactant on the Caco-2 permeability of lercanidipine.

FIG. 4 depicts the effect of various excipients on the dissolution profile of immediate release lercanidipine pharmaceutical bead compositions of the present invention, the curve represented by -∘- depicts the dissolution profile of an immediate release composition without surfactant and a bead size of 18-20 mesh drug loading of mg per g, --∘-- depicts the dissolution profile of an immediate release composition without surfactant and a bead size of 18-20 mesh with drug loading of mg per g, the curve represented by -♦- depicts the dissolution profile of an immediate release composition comprising Polysorbate 80 as a surfactant and a bead size of 18-20 mesh, the curve represented by -□- depicts the dissolution profile of an immediate release composition comprising PEG 400 as a surfactant and a bead size of 18-20 mesh, the curve represented by -x- depicts the dissolution profile of an immediate release composition comprising glycerin as a surfactant and a bead size of 18-20 mesh, the curve represented by -●- depicts the dissolution profile of an immediate release composition without surfactant and a bead size of 20-25 mesh, the curve represented by -Δ- depicts the dissolution profile of an immediate release composition comprising Explotab as a disintegrant and a bead size of 20-25 mesh, and the curve represented by -□- depicts the dissolution profile of an immediate release composition comprising PEG 400 as a surfactant and a bead size of 20-25 mesh.

FIG. 5 depicts the effect of seal coating on the dissolution profile of lercanidipine immediate release pharmaceutical bead compositions of the present invention, the curve represented by -x- depicts the dissolution profile of an immediate release composition without seal coating, the curve represented by -x- depicts the dissolution profile of an immediate release composition comprising seal coating to a 10% weight gain, the curve represented by -●- depicts the dissolution profile of an immediate release composition comprising seal coating to a 12% weight gain, the curve represented by -|- depicts the dissolution profile of an immediate release composition comprising seal coating to a 15% weight gain.

### DETAILED DESCRIPTION OF THE INVENTION

As used herein, the following terms are defined as follows:

The term “about” means within 10% of a given value and preferably within 5%, of a given value. Alternatively, the term “about” means that a value can fall within a scientifically acceptable error range for that type of value, which will depend on how qualitative a measurement can be given the available tools.

The phrase “dissolution profile” as used herein, refers to the dissolution of an agent over time. The dissolution can be measured as the relative amount of agent dissolved over time, the amount of agent dissolved, or the concentration of the agent. The preferred method of determining dissolution rate is USP basket method at 100 RPM in 900 ml aqueous buffer 0.01N HCl, at 37° C. Alternative methods are equally acceptable including the USP paddle method and other suitable methods known to those of skill in the art.

The term “lercanidipine” means the free base composition methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate, as well as any pharmaceutically acceptable salt, e.g., a salt with an inorganic or organic acid such as, HCl, HBr, H₂SO₄, maleic acid, fumaric acid, tartaric acid and citric acid. Preferred pharmaceutically acceptable salts of lercanidipine include, but are not limited to, hydrochloride, besylate and napadisylate salts. Additionally, lercanidipine may be present in crystalline and/or amorphous forms. Preferred pharmaceutically acceptable salts of lercanidipine include may be either R or S enantiomers, or a racemic mixture thereof.

The term “immediate release” means any type of release of the active ingredient, lercanidipine, from the composition of the present invention resulting in in-vitro release over a short period of time, i.e., (less than one hour) sufficient to provide therapeutically effective plasma levels over similarly short time interval and/or to modify other pharmacokinetic properties of the active ingredient. Preferably, the release of lercanidipine provides for a maximum concentration of lercanidipine (Cₘₐₓ) of about 12 ng/mL and a time to maximum plasma concentration (Tₘₐₓ) of about 1 to 3 hours for a 40 mg dose of lercanidipine.

As used herein, the term “pharmaceutically acceptable” refers to a biologically or pharmacologically compatible for in vivo use, and preferably means approved by a regulatory agency of the Federal or a state government or listed in the U.S. Pharmacopoeia or other generally recognized pharmacopoeia for use in animals, and more particularly in humans.

The term “bioavailability” refers to the rate and extent to which the active ingredient or active moiety, e.g., lercanidipine, is absorbed from a drug product, i.e., bead, and becomes available at the site of action, i.e., smooth muscle walls of arteries.

The terms “treat” and “treating” refer to reducing or relieving hypertension, e.g., decreasing either systolic or diastolic blood pressure in a patient by at least 10 mm Hg.

As used herein, a “therapeutically effective amount” refers to the amount of an active agent sufficient to lower the blood pressure of a patient with hypertension, e.g., the blood pressure is decreased by at about 15 mm Hg for systolic pressure or by about 10 mm Hg for diastolic pressure. A therapeutically effective amount of the active agent may or may not decrease the blood pressure in a person that does not have hypertension or may not decrease blood pressure in all persons with hypertension. In a preferred embodiment, the active agent decreases a patient's blood pressure to below 140/90 mm Hg. Treatment of other pathologies, such as heart failure or arthrosclerosis is also specifically contemplated as per, e.g., U.S. Pat. Nos. 5,696,139 and 5,767,136.

All weights and weight ratios specified for lercanidipine and pharmaceutically acceptable salts thereof are based on the weight of a molar equivalent of the hydrochloride salt of lercanidipine.

Pharmaceutical Compositions

The immediate release composition of the present invention is designed to provide for immediate release of lercanidipine upon exposure to an use environment, such as gastric fluid, upon administration. The immediate release composition of the present invention provides for both rapid dissolution of lercanidipine upon introduction of the composition to an aqueous environment, and for a rapid rise in plasma concentration of lercanidipine to therapeutic levels following administration to a patient.

The immediate release composition of the present invention comprises (i) an inert core and (ii) a first layer comprising lercanidipine, a surfactant and a binder and (iii) optionally a second layer comprising a film coating. The immediate release core may include additional excipients to improve appearance, handling and processing properties and/or dissolution properties of the active ingredient. Additional excipients contemplated by the present invention include, but are not limited to, carriers, diluents, lubricants, disintegrants, glidants and/or anti-adherent agents.

The inert core may comprise any pharmaceutically acceptable material, including, but not limited to, inorganic or organic non-pareil seeds, such as those made from microcrystalline cellulose, sugar or starch. Preferably the inert core has a mean size from about 10 and about 60 mesh, and more preferably from about 18 to 35 mesh Optionally, inert core with a mean size from about 35 and about 140 mesh can also be used for compression of beads into tablets. Preferably the ratio of the mass of the inert core to the mass of lercanidipine is from about 5:1 to about 20:1 and more preferably from about 5:1 to about 15:1.

The inert core is coated with a first layer comprising lercanidipine, a surfactant, and a binder. In one preferred embodiment, the lercanidipine is lercanidipine hydrochloride. Additionally, lercanidipine may be present in crystalline or amorphous forms and mixture thereof. Lercanidipine present in the crystalline form may be present in any polymorphic form or mixtures thereof, including those disclosed in U.S. Published Application Nos. 2003/0083355 and 2003/0069285 which are incorporated herein by reference. Preferred pharmaceutically acceptable polymorphs of lercanidipine are crystalline Form I and Form II. Additionally, lercanidipine may be amorphous or a mixture of amorphous and crystalline forms, wherein the crystalline can be of the same polymorph or a combination of two or more polymorphs.

One skilled in the art will appreciate that the immediate release compositions of the present invention may include one or more forms of lercanidipine, e.g., different salt forms, amorphous forms or crystalline forms, in order to achieve the desired in vitro dissolution profile and/or the desired in vivo plasma concentration of lercanidipine. In one embodiment, one skilled in the art may combine crystalline lercanidipine Forms I and II to achieve desired properties based upon bioavailability studies in dogs described in US. Published Application 2003/0083355 (herein incorporated by reference) that found lercanidipine crystalline polymorph Form II to have a higher bioavailability than lercanidipine crystalline polymorph Form I. Studies have also indicated, however, that Form I has a shorter time to maximum concentration attainable compared to Form II and that Form II has a higher plasma concentration (AUCo-t) and a delayed time of maximum concentration (Tₘₐₓ) compared to Form I. The novel present invention incorporates sufficient solubility/permeability enhancer surfactant that allows for the use of different polymorphs.

Preferably, lercanidipine is present in an amount sufficient to render a therapeutic effect when the immediate release composition of the present invention is administered to a patient. Lercanidipine may be present in any amount from about 0.001 to about 0.2 mg per mg of the total composition, and more preferably from about 0.005 mg to about 0.15 mg per mg of the total composition and most preferably 0.01 mg about 0.1 mg per mg of the total composition.

In addition to lercanidipine, the first layer coating the inert core preferably comprises a surfactant. Surfactants may be incorporated in the beads of the present invention to facilitate the wetting of lercanidipine and promote its adhesion to the inert core and/or binders. Surfactants may also be incorporated for the purpose of enhancing or modulating the solubility and permeability of lercanidipine in the environment of use.

Surfactants of the present invention include, but are not limited to anionic and non-ionic surfactants such as sodium lauryl sulfate, poloxamers (copolymers of polyoxyethylene and polyoxypropylene), natural or synthetic lecitins as well as esters of sorbitan and fatty acids, such as Span® (Commercially available from Sigma-Aldrich Co., St. Louis, Mo.), esters of polyoxyethylenesorbitan and fatty acids, such as Polysorbates or Polysorbate® (Commercially available from Spectrum Chemical, Gardena Calif.) polyoxyethylated hydrogenated castor oils, such as Cremophor® (Commercially available from BASF, Mount Olive, N.J.), polyoxyethylene stearates, such as Myrj® (Commercially available from Uniqema, New Castle, Del.) or any combinations of the surfactants. Preferably the surfactant is a polysorbate and most preferably the surfactant is Polysorbate 80 (Commercially available from Chemical, Gardena Calif.)., Vitamin E TPGS (Eastman Chemical Company, Kingsport, Tenn.)

The amount of surfactant may be adjusted, so as to moderate the solubility, permeability, and bioavailability of lercanidipine. Preferably the ratio of surfactant to lercanidipine on a mass basis is from about 0.001:1 to about 1:1, more preferably from about 0.005:1 to 0.6:1 and most preferably from about 0.01:1 to about 0.25:1.

The first layer coating of the inert core further preferably comprises a binder. Binders are incorporated in the beads of the present invention to facilitate the adhesion of lercanidipine to the inert core. Preferably, the binder does not interfere with or decrease the solubility of lercanidipine. Suitable binders include, but are not limited to, either individually or in combination, such binding agents and adhesives as sucrose; gelatin; glucose; starch; cellulose materials such as, but not limited to, methylcellulose and sodium carboxymethylcellulose ; alginic acid and salts of alginic acid; magnesium aluminum silicate; polyethylene glycol; guar gum; polysaccharide acids; bentonites; polyvinylpyrrolidone (povidone); polymethacrylates (such as Eudragit ); hydroxypropyl methylcellulose (HPMC); hydroxypropyl cellulose (Klucel™); ethyl cellulose (Ethocel™); pregelatinized starch (such as National™ 1511 and Starch 1500).

Preferably the binder comprises hydroxypropylmethyl cellulose and most preferably Opadry™ (commercially available from Colorcon, Inc., West Point, Pa.). Preferably the ratio of binder to lercanidipine on a mass basis is from about 0.01:1 to about 1:1, more preferably from about 0.1:1 to about 0.5:1.

Optionally the immediate release core may comprise a second layer comprising a film coating to improve the durability, appearance and/or handling of the bead composition. Preferably the film coating does not interfere with the dissolution and/or pharmacokinetic properties of the bead composition of the present invention. Examples of film coatings contemplated by the present invention include, but are not limited to, those that include hydroxypropylmethyl cellulose and particularly Opadry™. and polymethacrylates (Eudragits™). However, any film-former known in the art may be used.

Preferably, the first layer and optional second layer are applied such that the immediate release beads have an average radius from about 2 to about 0.1 mm (10 to 140 mesh) and more preferably from about 1.4 to about 0.5 mm (14 to 35 mesh).

One skilled in the art will appreciate that the rate of lercanidipine release from the immediate release bead composition may be controlled by factors such as the composition, surfactant content, and binder content of the immediate release core, the thickness and permeability of the film coating and the surface area-to-volume ratio of the beads themselves. It will be appreciated by those skilled in the art that increasing the thickness of the coating will decrease the release rate, whereas increasing the permeability of the coating or the surface area-to-volume ratio of the beads will increase the release rate.

Moreover, it will be appreciated by those skilled in the art that the desired in vitro dissolution rate, and/or the in vivo plasma concentration of lercanidipine over time, may be obtained by selecting one or more forms of lercanidipine, i.e., selecting one or more salt forms, crystalline forms (including one or more polymorphic forms) or amorphous forms for use in the immediate release compositions of the present invention.

Optionally, the pharmaceutical compositions of the present invention may include additional excipients to improve appearance, handling and processing properties and/or dissolution properties of the active ingredient. Additional excipients contemplated by the present invention include, but are not limited to, carriers, dilutents, disintegrants, lubricants, glidants and/or anti-adherent agents.

Suitable lubricants and/or glidants include, but are not limited to, either individually or in combination, such lubricants and/or glidants as glyceryl behenate (Compritol™ 888); metallic stearates (e.g., magnesium, calcium and sodium stearates); stearic acid; hydrogenated vegetable oils (e.g., Sterotex™); talc; waxes; Stearowet™; boric acid; sodium benzoate and sodium acetate; sodium chloride; DL-Leucine; polyethylene glycols (e.g., Carbowax™ 4000 and Carbowax™ 6000); sodium oleate; sodium benzoate; sodium acetate; sodium lauryl sulfate; sodium stearyl fumarate (Pruv™); and magnesium lauryl sulfate.

Additional suitable anti-adherents or glidants include, but are not limited to, either individually or in combination, such anti-adherents as talc, cornstarch, DL-Leucine, sodium lauryl sulfate, and metallic stearates.

Other carrier materials (such as colorants, flavors and sweeteners) and modes of administration are known in the pharmaceutical art and can be used in the preparation of the pharmaceutical compositions of the present invention. Moreover, it will be appreciated by those skilled in the art that the desired dosage from can be in the form of a capsule or a compressed tablet.

Manufacture of Pharmaceutical Compositions

The immediate release pharmaceutical bead composition of the present invention may be manufactured using any number of processes well known in the art. In one embodiment the compositions of the present invention may be prepared as beads by coating sugar spheres with an aqueous suspension containing lercanidipine followed by application of a film coating.

In one embodiment, sugar spheres are preheated in a fluidized bed coater (e.g., GPGC3, Glatt Air Technique, Ramsey, N.J.) for about 10 minutes and more preferably for about 5 minutes, between product temperatures of about 30° C. and about 50° C. and more preferably between about 36° C. and 46° C. Drug loading may be carried out using any method known in the art, such spray coating, although other coating methods may be used. Preferably, the preheated sugar spheres are coated with a suspension containing lercanidipine, a binder, a surfactant and purified water in a fluidized bed coater using a spray pressure between about 1.5 and 2.5 bars, at a product temperature between 30° C. and about 50° C. and more preferably between about 36° C. and 42° C. Optionally, other methods including pan coating is equally acceptable.

Drug loaded beads may optionally be film coated by coating the beads with an aqueous dispersion material such as Opadry™. An aqueous film coating dispersion may be applied using any method known in the art, such as spray coating the beads in a fluidized bed coater at a spray pressure between about 1.5 and 2.0 bars and a product temperature between about 30° C. and about 55° C. and more preferably between about 41° C. and 47° C.

Following drug loading and/or film coating, the beads may be dried and cured. One skilled in the art will appreciate that drying and curing conditions will vary depending upon several factors including, solvents, the size of the substrate, type and level of binder, type of level of surfactants, the thickness of the coating, and the amount of material in the composition. In one embodiment, the immediate release beads are dried in a fluidized bed for about 10 minutes and more preferably for about 5 minutes, between about 35° C. and about 60° C. and more preferably between about 40° C. and 50° C.

Unit Dosage Forms

To form oral unit dosage forms, dried beads may be combined and loaded into gelatin capsules, or other delivery devices suitable for oral administration. The drug layered beads may be combined with additional excipients, such as mannitol, starch, HPMC, magnesium stearate and compressed into a tablet to create a solid oral dosage form. Preferably, the unit dosage forms comprise a sufficient amount of the immediate release beads of the present invention to impart a therapeutic effect when the dosage form is administered to a patient. More preferably, the unit dosage form comprises from about 1 to about 80 mg of lercanidipine, and most preferably about 5 to about 80 mg of lercanidipine immediate release composition.

In one embodiment the immediate release compositions of the present invention may be combined with one or more modified release compositions to yield a unit dosage exhibiting a multi-phase release profile. Preferably the unit dosage form comprises both immediate and modified release compositions and comprises a total dosage of lercanidipine of about 1 to about 80 mg and more preferably about 5 to about 80 mg of lercanidipine, wherein from about 5 to about 40 mg of the lercanidipine is provided as an immediate release composition. In another embodiment, the immediate release compositions of the present invention may be combined with additional active agents.

Preferably the ratio of immediate release to modified release compositions is such that the dosage form, when administered to a patient, provide both rapid and longer term relief from hypertension. Preferably, the ratio of immediate to modified release compositions is such that the dosage form provide for maximum plasma concentration of lercanidipine from about 10 to about 14 ng/mL and therapeutic plasma concentrations of lercanidipine for at least about 24 hours. In one embodiment the ratio of immediate release to modified release compositions is preferably from about 1:1 and 1:50, more preferably from about 1:2 and 1:20 and most preferably from about 1:5 and 1:10.

Dissolution Profile

The immediate release compositions of the present invention are designed to produce a rapid rise to therapeutic plasma levels of lercanidipine after oral administration, due to the rapid dissolution and increased permeability of lercanidipine. Both the dissolution of substance in the gastrointestinal fluid and its permeation are required to ensure sufficient bioavailability. The dissolution properties and permeability of the composition of an active agent are important in evaluating its ability to be absorbed and made available at the site of action. Therefore, when evaluating the potential bioavailability of an active agent, it is important to determine the dissolution profile of the composition.

The dissolution profile for an active agent from a dosage unit is determined as the proportion of the amount of active agent released from the dosage unit over a specified time. The test method used references the results, so it is important to specify the method as well as the conditions under which measurements were made. Preferably the dissolution properties of the immediate release compositions of the present invention are determined using the dissolution method, (1) USP basket method at 100 RPM in 900 ml aqueous buffer 0.01N HCl, at 37° C. Alternate methods such as those described in the USP, e.g., paddle method at 50 RPM in 900 ml aqueous buffer 0.1N HCl with Polysoabate 80 at 37° C., are equally acceptable.

With the above in mind, the in vitro dissolution of lercanidipine at various time points for compositions in accordance with the present invention is preferably about more than 80% dissolved within about the first 60 minutes, more preferably at least more than about 80% of dissolved within about the first 30 minutes, and still more preferably at least about 50% dissolved within about the first 15 minutes.

In other embodiments, dissolution of lercanidipine composition may be evaluated using the USP paddle method at 50 RPM in 900 ml FaSSIF buffer, at 37° C. One skilled in the art will appreciate that the amount dissolved will be dependent on the specific dissolution conditions.

Pharmacokinetic Profiles

In addition to providing for rapid dissolution of lercanidipine, it is an objective of the present invention to provide an immediate release composition having a pharmacokinetic profile which provides for rapid onset of, blood pressure lowering while avoiding undesirable side-effects. Such a pharmacokinetic profile provides for a rapid rise in lercanidipine plasma concentration following administration to a patient, e.g., from about 6 to about 14 ng/ml of lercanidipine, followed by a steady decline in plasma concentration to a level from about 0.4 to 0.1 ng/ml of lercanidipine. Preferably, the pharmacokinetic profile does not have any erratic peaks or troughs, but rather provides for a steady and consistent rise in lercanidipine concentration to therapeutic levels, followed by a steady and consistent decline.

Additionally, it is an objective of the present invention to provide an immediate release composition which shortens the time to maximum plasma concentration (Tₘₐₓ), relative to commercially available immediate release lercanidipine capsules or tablets. Preferably, upon administration of the immediate release composition of the present invention to a patient, the Tₘₐₓ is from about 0.5 to about 3 hours.

Treatment of Specific Conditions and Disorders

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity and a decrease in blood pressure while minimizing toxicity or side-effects for a particular patient. However, such fine tuning of the therapeutic regimen is routine in light of the guidelines given herein.

The dosage of the immediate release composition of the present invention may vary according to a variety of factors such as underlying disease state, the individual's condition, weight, sex and age and the mode of administration. For oral administration, the pharmaceutical compositions can be provided in the form of scored or unscored unit dosage forms or capsules.

In one embodiment for the treatment of hypertension, the pharmaceutical composition or oral dosage form comprising immediate release beads of the present invention preferably comprises from about 2 to 80 mg lercanidipine. More preferably, the composition or dosage form comprises from about 2 to 80 mg lercanidipine.

The pharmaceutical composition or unit dosage form may be administered in a single daily dose, or the total daily dosage may be administered in divided doses. In addition, co-administration or sequential administration of other active agents may be desirable. The immediate release compositions of the invention may be combined with any known drug therapy, preferably for treatment of hypertension. For example, bimodal therapy involving in addition a diuretic, a β-receptor blocker, an ACE inhibitor or an angiotensin II receptor antagonist is contemplated by the present invention (see, e.g., U.S. patent application Ser. No. 10/791,148, which is hereby incorporated by reference.)

The immediate release compositions of the current invention may be combined with additional active agents. Two different 1,4-dihydropyridines may be used, or the lercanidipine may be combined with other active agents or other therapies. For example, an immediate release composition of the present invention may be combined with an ACE inhibitor, such as enalapril, described in U.S. Patent Publication No. 2003/00180355, or with lisinopril as described in commonly-owned U.S. patent application Ser. Nos. 10/688,061 and 10/829,932. Lercanidipine may also be combined with an angiotensin II receptor blocker (ARB) such as irbesartan or olmesartan (U.S. patent application Ser. No. 10/791,148). Also contemplated by the present invention is addition of a diuretic or a receptor blocker to the lercanidipine formulation. Exemplary diuretics include thiazide diuretics, potassium sparing diuretics, loop diuretics, such as hydrochlorothiazide, spironolactone, and ethacrynic acid, respectively.

Non-limiting examples of ACE inhibitors include benazepril, captopril, cilazapril, enalapril, fentiapril, fosinopril, indopril, lisonopril, moexipril, perindopril, quinapril, ramipril, spirapril and their pharmaceutically acceptable salts. Exemplarily diuretics include furosemide, hydrochlorothiazide, torasemide, indapamide and eplerenone. Exemplarily CCB's include amlodipine, nifedipine and verapamil. Exemplarily beta blockers include atenolol, carvediol, nadolol and propranolol. Exemplarily alpha blockers include clonidine and prazosin. Exemplarily ARBs include, candesartan, eprosartan, irbesartan, losartan, olmesartan, saprisartan, telmisartan, valsartan, E-1477, SC-52458, ZD-8731, and their pharmaceutically acceptable salts. Additional ARBS include those found in EP 443983, EP 253310, EP 403159, EP 420237, EP 502314, EP 504888, EP 514198, EP 475206, and WO 93/20816, each of which are herein incorporated by reference.

The immediate release compositions of the present invention may also be combined in a therapy with a second active agent, such as those described above, where the two agents are administered sequentially. Either the lercanidipine or the second agent may be delivered first, and the time between treatment of the lercanidipine and second agent may be for a period from about 1-2 hours, to about 2-6 hours, to about 6-12 hours, to about 12-24 hours following administration of the first agent. Similarly, this same time period may occur between a first and third agent in the case of a three-way combination. Alternatively, simultaneous administration of the 1,4-dihydropyrine and second active agent, with or without sequential administration of either the 1,4-dihydropyrine and second active agent could also be employed.

For combination therapy, the compounds may initially be provided as separate dosage forms until an optimum dosage combination and administration regimen is achieved. Therefore, the patient may be titrated to the appropriate dosages for his/her particular hypertensive condition. After the appropriate dosage of each of the compounds is determined to achieve a decrease of the blood pressure without untoward side effects, the patient than may be switched to a single dosage form containing the appropriate dosages of each of the active agents, or may continue with a dual dosage form.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of compounds within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

### EXAMPLES

The following examples of immediate release compositions and methods of making the same are now disclosed. The following examples are illustrative in nature of the various aspects of the present invention and are not intended to be limiting in any manner.

### Example 1

### Preparation of Lercanidipine Immediate Release Beads

An immediate release composition comprising 10 mg of lercanidipine was prepared having the composition shown in Table 1.

TABLE 1
10 mg lercanidipine immediate release bead composition

| Ingredient | mg/capsule | Weight % Composition |
|---|---|---|
| Lercanidipine HCl | 10 | 12.26 |
| Polysorbate 80, NF | 0.75 | 0.92 |
| Sugar Spheres, USP | 66.75 | 81.80 |
| Opadry Clear (Binder Portion) | 2.50 | 3.06 |
| Opadry Clear (Film Coating | 1.60 | 1.96 |
| Portion) | | |

The immediate release composition of the present example was prepared by loading approximately 8.18 kg sugar spheres, USP (Parlar Corp., Cranbury N.J. having a size of approximately 20-25 mesh into a GPCG5 fluidized bed coater. The sugar spheres were preheated for about 5 minutes between 34 and 44° C.

The preheated spheres were spray coated with an aqueous lercanidipine suspension in a GPCG5 fluidized bed coater, using a Wuster Coating, Glatt Air Technique, Ramsey, N.J. at a spraying pressure between 1 and 3 bars and a temperature between 34 and 44° C. The lercanidipine suspension was prepared by first preparing a suspension of Opadry™ Clear by mixing 0.306 Kg Opadry™ Clear (Colorcon, Inc, West Point, Pa.) in 11.6 L purified water with continuous stirring until fully dissolved. To a portion of the Opadry suspension 0.092 Kg Polysorbate 80 [Spectrum , New Brunswikc, N.J.] was added with continuous stirring followed by the addition of 1.226 Kg lercanidipine HCl (Recordati SpA, Milan, Italy). Once the lercanidipine HCl was fully dispersed, the remaining Opadry™ Clear was added to complete the suspension.

Following drug loading the beads were film coated by coating with Opadry™ Clear. A aqueous dispersion of Opadry™ Clear was prepared by mixing 0.196 Kg Opadry™ Clear with 2.45 L purified water with continuous stirring until the Opadry™ Clear was completely dissolved. The film coating solution was applied by spraying the beads in a fluidized bed coater using a spray pressure between about 1 and 3 bars, at a temperature between about 34 and 44° C.

Film coated beads were dried in a fluidized bed for about 5 minutes between about 34 and 44° C. Optionally, multiple sub lots of beads were mixed in a V-blender and stored sealed under suitable conditions.

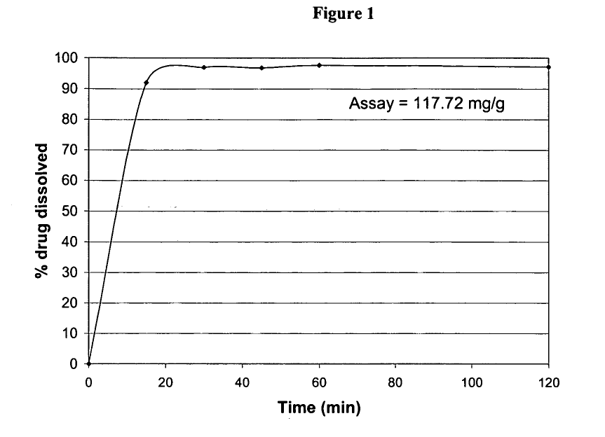

Immediate release beads prepared as described in the present example were subjected to in vitro dissolution analysis. Dissolution analysis was carried out via the USP I basket method, in 900 ml 0.01 N hydrochloric acid containing at 37° C., 100 RPM. The dissolution results are set forth in Table 2 below and are depicted in FIG. 1. FIG. 1 shows that more than 90% of the lercanidipine was dissolved within a period of about 30 minutes following introduction of the immediate release composition to the aqueous medium.

TABLE 2
Dissolution results of Example 1: 10 mg lercanidipine
immediate release bead compositions

| | Percent lercanidipine |
|---|---|
| Time (min) | dissolved (%) |
| 0 | 0 |
| 15 | 92 |
| 30 | 97 |
| 45 | 97 |
| 60 | 98 |
| 120 | 97 |
| 150 | 97 |

### Example 2

### Effect of Surfactant on In Vitro Dissolution and Permeability

To determine the effect of surfactant on the in vitro dissolution profile, immediate release lercanidipine beads were prepared and subjected to dissolution analysis using the dissolution method described in Example 1.

Lercanidipine immediate release beads were prepared as described below in order to assess the effect of surfactant on dissolution properties. The immediate release beads of the present example were prepared having the compositions shown in Table 3.

TABLE 3
Immediate release bead composition with and without surfactant

| | Formulation A | Formulation B |
|---|---|---|
| Ingredient | (mg/capsule) | (mg/capsule) |
| Lercanidipine HCl | 20 | 20 |
| Polysorbate 80, NF | 0 | 1.4 |
| Sugar Spheres, USP | 326 | 324.6 |
| Opadry Clear (Binder) | 4 | 4 |
| Opadry Clear (Film Coating) | 1.6 | 1.6 |

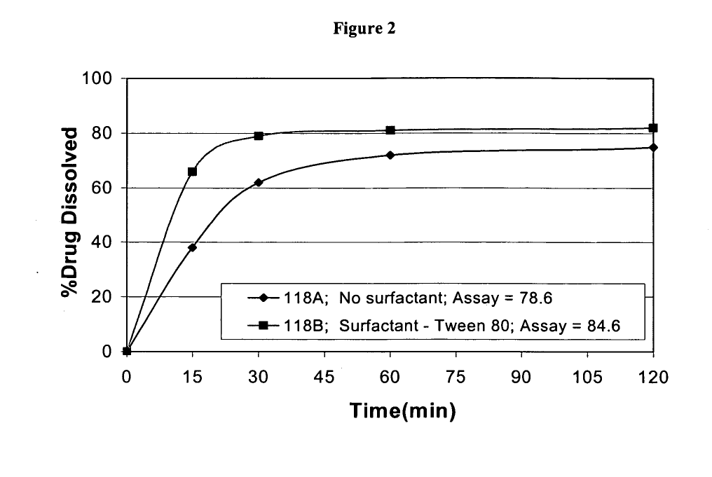

The beads were manufactured, coated and cured in the same manner as described in Example 1. The dissolution profiles of the beads prepared in the present example were determined using the USP basket method as described in Example 1. The dissolution results are set forth in Table 4 below and depicted in FIG. 2. FIG. 2 illustrates that the surfactant acts to solubilize lercanidipine and thereby increases its dissolution rate.

TABLE 4
Dissolution results of Example 2 immediate
release bead compositions

| | Formulation A % | Formulation B % |
|---|---|---|
| Time | lercanidipine | lercanidipine |
| (min) | dissolved | dissolved |
| 0 | 0 | 0 |
| 15 | 49 | 80 |
| 30 | 78 | 92 |
| 60 | 91 | 95 |
| 120 | 97 | 96 |

Caco-2 Cell Analysis

The objective of this study was to investigate the effects of surfactant on the permeability characteristics of lercanidipine across Caco-2 cell monolayers. Caco-2 cell monolayers have been used as a model of intestinal mucosa for predicting oral drug absorption (P. Artursson. Epithelial transport of drugs in cell culture. I: A model for studying the passive diffusion of drugs over intestinal absorptive (Caco-2) cells. JPharm (1990)).

Transport experiments for the permeation characteristics of lercanidipine in the presence of different concentrations of Polysorbate 80 (Tween 80) were conducted as follows. Five hundred μl of dosing solution was added to the donor (apical) compartment of Caco-2 cell monolayers grown for 21 in Transwell system and 1500 μl of fresh Hank's Balanced Salt Solution (HBSS) containing 1% of bovine serum albumin (BSA) were placed in the receiver (basolateral) compartment. The samples at the receiver side were taken at time 100 min. Aliquots (20 μl) were withdrawn from the donor side at 0 and 10 min. The samples were collected and analyzed for lercanidipine.

The permeation characteristics lercanidipine across Caco-2 cell monolayers in the presence of different concentrations of Tween 80 were analyzed using four dosing solutions. The pH value for dosing solutions was 6.8. The four treatments were: (A) 50 μg/ml of Lerc in HBSS containing no Tween 80, (B): 50 μg/ml of Lerc in HBSS containing 0.002% (w/v) Tween 80, (C): 50 μg/ml of Lerc in HBSS containing 0.02% Tween 80, and (D): 50 μg/ml of Lerc in HBSS containing 0.2% Tween 80.

Calculation

Pₐₚₚ values will be calculated using the equation:
Pₐₚₚ=ΔQ/Δt/(A*C₀)
where ΔQ/Δt is the linear appearance rate of mass in the receiver solution, A is the filter/cell surface area (1 cm² for 12-well system), and C₀ is the initial concentration of the test compounds.

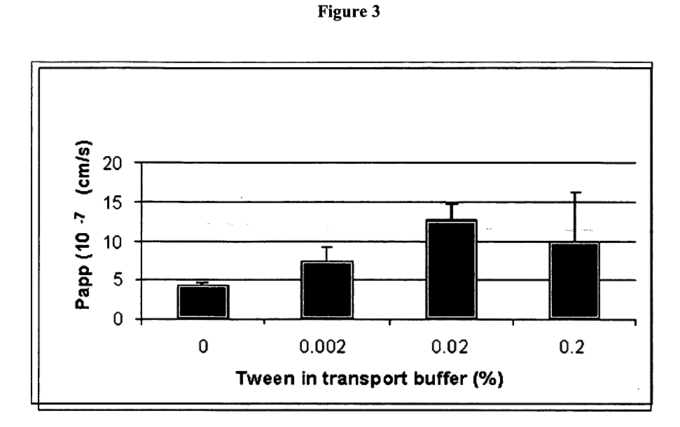

The results of the present Caco-2 cell experiment are depicted in FIG. 3 and confirm that the permeability of lercanidipine is enhanced by the presence of surfactant. In the present example, lercanidipine displayed a three fold increase in permeability upon the addition of surfactant. In the present example, the surfactant acts as a solubilizer and increases the dissolution and permeability of lercanidipine. The effect of the surfactant, however, is not without limitation, as when the amount of surfactant in the transport buffer is increased ten fold from 0.02 to 0.2, the permeability of lercanidipine decreases.

### Example 3

### Comparative Example to Determine the Effect of Excipients on In Vitro Dissolution

To determine the effect of excipients on the in vitro dissolution profile of lercanidipine immediate release beads, beads having the compositions shown in Table 5 were prepared. The in vitro dissolution properties of the beads prepared according to Table 5 were compared to the dissolution properties of beads prepared according to Example 1 as described below.

TABLE 5
Immediate release bead composition with and without
surfactant. All values provided as mg/capsule

| | Formulation | | | | | |
|---|---|---|---|---|---|---|
| Ingredient | C | D | E | F | G | H |
| Lercanidipine HCl | 40 | 40 | 40 | 40 | 40 | 40 |
| Polysorbate 80, NF | 0 | 0 | 0 | 0 | 0 | 0 |
| PEG 400 | 0 | 1.462 | 0 | 0 | 0 | 1.462 |
| Glycerin | 0 | 0 | 1.462 | 0 | 0 | 0 |
| Explotab | 0 | 0 | 0 | | 1.462 | |
| Sugar Sphere, USP | 272 | 270.5 | 270.5 | 272 | 270.5 | 270.5 |
| Sugar Sphere Mesh | 18-20 | 18-20 | 18-20 | 20-25 | 20-25 | 20-25 |
| Size | | | | | | |
| Opadry Clear | 8 | 8 | 8 | 8 | 8 | 8 |
| (Binder) | | | | | | |

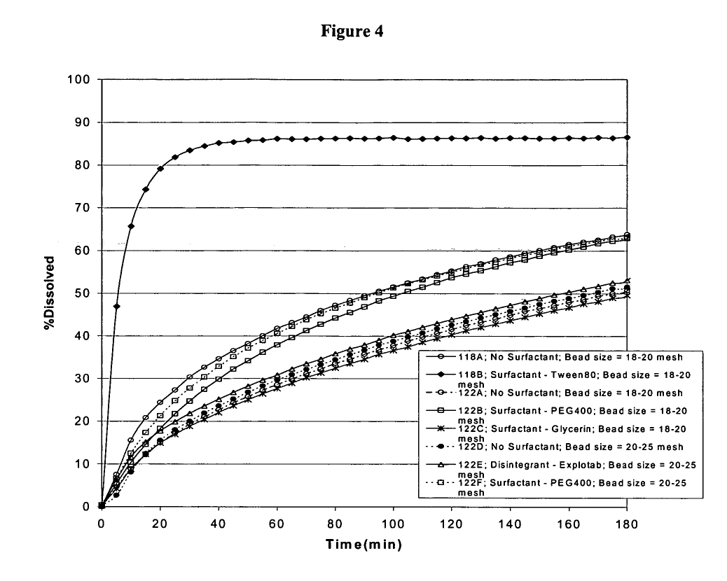

The beads were manufactured, coated and cured in the same manner as described in Example 1 with the exception that the optional second coating was not performed. The dissolution profiles of the beads prepared in the present example were determined using the USP basket method as described in Example 1. The dissolution results are set forth in Table 6 below and depicted in FIG. 4. The results of the present example indicate that the presence of surfactant in the compositions enhance the solubility of lercanidipine and that the most rapid and greatest solubility is achieved without the presence of additional excipients.

TABLE 6
Dissolution results of Example 3 immediate release bead compositions
compared to immediate release bead compositions of Example 1.
Values provided as % of lercanidipine dissolved.

| | | Formulation | | | | | |
|---|---|---|---|---|---|---|---|
| Time (min) | Example 1 | C | D | E | F | G | H |
| 0 | 0 | 0 | 0 | 0 | 0 | 0 | 0 |
| 15 | 88 | 15 | 20 | 12 | 21 | 15 | 18 |
| 30 | 97 | 24 | 30 | 19 | 31 | 21 | 28 |
| 45 | 100 | 32 | 37 | 22 | 37 | 26 | 35 |
| 60 | 100 | 38 | 42 | 27 | 40 | 31 | 41 |
| 120 | 100 | 53 | 56 | 41 | 54 | 44 | 55 |
| 180 | 100 | 63 | 64 | 50 | 63 | 52 | 63 |

### Example 4

### Effect of Film Coating on In Vitro Dissolution

To determine the effect of film coating on the in vitro dissolution profile, lercanidipine immediate release beads having the compositions shown in Table 7 were prepared.

TABLE 7
Immediate release bead formulation having varies amounts
of film coating. All values provided as mg/capsule

Formulation

| Ingredient | I | J | K | L |
|---|---|---|---|---|
| Lercanidipine HCl | 40 | 40 | 40 | 40 |
| Polysorbate 80, NF | 23 | 23 | 23 | 23 |
| Sugar Sphere, USP | 340 | 340 | 340 | 340 |
| Opadry Clear (Binder) | 10 | 10 | 10 | 10 |
| Opadry Clear (Film | 0 | 6.8 | 17 | 34 |
| Coating) | | | | |

The beads were manufactured, coated and cured in the same manner as described in Example 1. The dissolution profiles of the beads prepared in the present example were determined using the paddle method with the USP apparatus II in FaSSIF media at 50 rpm and 37° C.

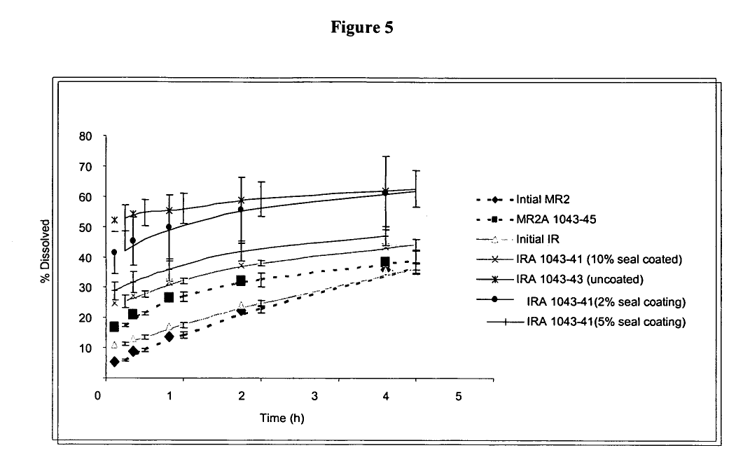

Fasted state simulated small intestinal fluid (FaSSIF) buffer was prepared in the laboratory as described in J. B. Dressman, G. L. Amidon, C. Reppas and V. P. Shah, “Dissolution testing as a prognostic tool for oral drug absorption: immediate release dosage forms”, Pharm Res. 15:11-22 (1998). FaSSIF buffer has previously been used as the bio-relevant buffer to predict the in vivo performance of an orally administered dosage form (J. B. Dressman, G. L. Amidon, C. Reppas and V. P. Shah, “Dissolution testing as a prognostic tool for oral drug absorption: immediate release dosage forms”, Pharm Res. 15:11-22 (1998)). Therefore, the dissolution studies were conducted in FaSSIF buffer in a USP apparatus II (50 rpm, 37° C.). The dissolution results are set forth in Table 7 below and depicted in FIG. 5.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate and are provided for description.

Patents, patent applications, publications, product descriptions, and protocols are cited throughout this application, the disclosures of which are incorporated herein by reference in their entireties for all purposes.

What is claimed is:

1. An immediate release solid dosage form comprising about 1 mg to 80 mg of lercanidpine wherein the solid oral dosage form has an in vitro release rate for the lercanidipine of more than about 80% within about the first 60 minutes following entry of the solid oral dosage form into a use environment and wherein the solid oral dosage form exhibits an average Tₘₐₓ within the range of about 0.5 hour to about hours 4 hours after entry of the solid dosage form into a use environment.

2. The immediate release solid dosage form according to claim 1 wherein the solid oral dosage form releases in-vitro the lercanidipine at a rate of more than about 80% within the first 30 minutes following entry of the solid oral dosage form into a use environment.

3. The immediate release solid dosage form according to claim 1 wherein the solid oral dosage form releases the lercanidipine at a rate of more than about 50% within the first 15 minutes following entry of the solid oral dosage form into a use environment.

4. The immediate release solid dosage form according to claim 1 wherein the solid oral dosage form exhibits an average Tₘₐₓ within the range of about 0.5 hour to about 3 hours after entry of the solid dosage form into a use environment.

5. The immediate release solid dosage form according to claim 1, wherein the lercanidipine is selected from the group consisting of lercanidipine hydrochloride, lercanidipine besylate and lercanidipine napadisylate.

6. The immediate release solid dosage form according to claim 1, wherein the lercanidipine is present in an amount ranging from about 2 mg to about 60 mg.

7. An immediate release pharmaceutical composition comprising:
(a) an inert core;
(b) a first layer substantially enveloping the core, comprising lercanidipine, a surfactant and a binder; and
(c) optionally comprising a second layer comprising an additional film coating.

8. The immediate release pharmaceutical composition of claim 7, wherein the lercanidipine is present in an amount from about 1% (10 mg per g) to about 80% (800 mg per g) of weight of the inert core.

9. The immediate release pharmaceutical composition of claim 7, wherein the ratio by weight of the lercanidipine to the inert core is from about 0.01:1 to about 0.8:1.

10. The immediate release pharmaceutical composition of claim 7, wherein the inert core comprises at least one material selected from the group consisting of microcrystalline cellulose, sucrose (sugar), mannitol, and starch.

11. The immediate release pharmaceutical composition of claim 7, wherein the inert core has a size from about 10 and about 140 mesh.

12. The immediate release pharmaceutical composition of claim 7, wherein the inert core is a tablet.

13. The immediate release pharmaceutical composition of claim 7, wherein the surfactant is selected from the group consisting of sorbitan and fatty acids, esters of polyoxyethylenesorbitan and fatty acids, polyoxyethylated hydrogenated castor oils, polyoxyethylene stearates, polaxomer, PEG, Vitamin E, and combinations of two or more thereof.

14. The immediate release pharmaceutical composition of claim 7, wherein the ratio by weight of surfactant to lercanidipine hydrochloride is from about 0.005:1 to about 0.6:1.

15. The immediate release pharmaceutical composition of claim 7, wherein the ratio by weight of the surfactant to lercanidipine hydrochloride is from about 0.01:1 to about 0.25:1.

16. The immediate release pharmaceutical composition of claim 7, wherein the binder is selected from the group consisting of polyvinylpyrrolidone, polymethacrylates; hydroxypropyl methylcellulose, hydroxypropyl cellulose, ethyl cellulose, pregelatinized starch and combinations of two or more thereof.

17. The immediate release pharmaceutical composition of claim 7, wherein the ratio by weight of the binder to lercanidipine is from about 0.01:1 to about 1:1.

18. The immediate release pharmaceutical composition of claim 7, wherein the ratio by weight of the binder to lercanidipine is from about 0.1:1 to about 0.5:1

19. The immediate release pharmaceutical composition of claim 7, wherein the film coating consists of one or more layers.

20. An immediate release oral dosage form comprising the immediate release pharmaceutical composition of claim 7 encapsulated within a capsule to form a solid oral dosage form.

21. An immediate release oral dosage form comprising the immediate release pharmaceutical composition of claim 7 compressed into a tablet to form a solid oral dosage form.

22. An immediate release oral dosage form comprising a plurality of immediate release lercanidipine beads, the immediate release beads comprising (i) lercanidipine, (ii) a core, (iii) a surfactant, (iv) a binder, and (iv) optionally, an additional film coating.

23. The immediate release oral dosage form according to claim 22 wherein the dosage form is administered to a mammal in need thereof.

24. The immediate release oral dosage form according to claim 23, wherein the mammal is a human.

25. The immediate release dosage form according to claim 1 wherein the average maximum plasma concentration of the lercanidipine is from about 0.5 to about 10 ng/ml, per 20 mg dose of the lercanidipine in a use environment in a human.

26. A method of treating hypertension in a patient in need thereof comprising orally administering an immediate release lercanidipine dosage form comprising (i) an inner core, (ii) a first layer comprising lercanidipine, a surfactant and a binder, and (iii) optionally, an additional film coating.

27. The method of claim 26 wherein administration of the immediate release lercanidipine dosage form to the patient results in a maximum plasma concentration of lercanidipine from about 10 to about 14 ng/ml, per 20 mg dose of lercanidipine.

28. The method of claim 26 wherein the time to the maximum plasma concentration is from about 10 to about 60 minutes after administration of the dosage form to a patient.

29. The immediate release oral dosage form according to claim 25, wherein the immediate release lercanidipine oral dosage form is present in amounts ranging from about 1% w/w to about 50% w/w.

30. The immediate release oral dosage form according to claim 29, wherein the immediate release lercanidipine oral dosage form is present in amounts ranging from about 2% w/w to about 15% w/w.

31. The immediate release pharmaceutical composition of claim 7, wherein the surfactant increases the permeability of the lercanidipine by more than 50% following entry of the composition into a use environment.

32. A method of measuring the increased permeability of the lercanidipine in the immediate release pharmaceutical bead composition of claim 29 comprising the use of a CaCo2 cell.

33. A pharmaceutical composition comprising beads, wherein each bead comprises:
(a) a core;
(b) a first layer substantially enveloping the core, comprising lercanidipine, a surfactant and a binder, and
(c) optionally, a second layer comprising a film coating,
wherein the dissolution rate of the lercanidipine in vitro is from about 50 to about 60% (by weight) dissolved within a period of about 15 minutes, from about 60 to about 70% (by weight) dissolved within a period of about 30 minutes and from about 70 to about 90% (by weight) dissolved within a period of about 45 minutes.

34. The pharmaceutical composition of claim 33, wherein the lercanidipine is present in the amount from about 0.001 to about 0.2 mg per mg of total weight of the composition.

35. The pharmaceutical composition of claim 33, wherein the ratio of the lercanidipine to the surfactant is from about 0.001:1 to about 0.2:1.

36. The pharmaceutical composition of claim 33, wherein the ratio of the lercanidipine to the binder is from about 0.01:1 to about 1:1.

37. The pharmaceutical composition of claim 33, wherein the ratio of the lercanidipine to the inert core is from about 0.01:1 to about 1:1.

38. The immediate release pharmaceutical composition of claim 33, wherein the surfactant is selected from the group consisting of sorbitan and fatty acids, esters of polyoxyethylenesorbitan and fatty acids, polyoxyethylated hydrogenated castor oils, polyoxyethylene stearates and combinations of two or more thereof.

39. The immediate release pharmaceutical composition of claim 33, wherein the core comprises at least one material selected from the group consisting of: microcrystalline cellulose, sugar and starch.

40. The immediate release pharmaceutical composition of claim 33, wherein the core has a size from about 10 and about 30 mesh.

41. The pharmaceutical composition of claim 33, wherein the binder is selected from the group consisting of polyvinylpyrrolidone, polymethacrylates; hydroxypropyl methylcellulose, hydroxypropyl cellulose, ethyl cellulose, pregelatinized starch and combinations of two or more thereof.

42. The immediate release pharmaceutical composition of claim 33, wherein the second layer comprises a film coating.

43. An immediate release oral dosage form comprising a plurality of immediate release lercanidipine beads, the immediate release beads comprising (i) lercanidipine, (ii) a core, (iii) a surfactant, (iv) a binder, and (iv) optionally, a film coating.

44. The immediate release oral dosage form of claim 43, wherein the immediate release lercanidipine beads are encapsulated within a capsule.
